(19) United States
(12) Patent Application Publication
(10) Pub. No.: US 2024/0245087 A1
(43) Pub. Date: Jul. 25, 2024

(54) Fermented Camel Milk with Propolis-Engineered Probiotic for Treating Adverse Bowel Disorders

(71) Applicant: KING FAISAL UNIVERSITY, AL-AHSA (SA)

(72) Inventors: ASHRAF KHALIFA, AL-AHSA (SA); HAIRUL ISLAM MOHAMED IBRAHIM, AL-AHSA (SA)

(21) Appl. No.: 18/100,626

(22) Filed: Jan. 24, 2023

(51) Int. Cl. A23L 33/135; A61K 31/353; A61K 35/20; A61K 35/644; A61K 35/744

(52) U.S. Cl. CPC A23L 33/135; A61K 31/353; A61K 35/20; A61K 35/644; A61K 35/744; A61K 2035/115

(57) ABSTRACT

The use of fermented camel milk with a honey propolis engineered probiotic for treating bowel disorders (BD). The honey propolis is used as a synergistic supplement to engineer the probiotic bacterium, Enterococcus faecium (EF), at a defined bacterial load, propolis concentration, and incubation period. The engineered EF is included with fermented camel's milk, which is then administered orally to those suffering from a BD. Effective treatment can be observed both through reduced adverse effects, as well as reduced levels of immune-modulatory markers such as TNF α, VEGF-1, IL-1β, IFN-γ IFN T γ-6 and TGF-β.

### FIELD AND BACKGROUND OF THE INVENTION

The disclosure of the present patent application relates to fermented camel milk compositions having propolis engineered probiotics. These compositions can be useful for treating various adverse bowel disorders.

### DESCRIPTION OF THE PRIOR ART

There is currently no effective and safe medical cure for treating various bowel disorders (BD). Rather, BD are typically treated pharmacologically by reducing inflammation and thus controlling gastrointestinal symptoms.

To treat severe symptoms, medical treatments such as 5-aminosalicylic acid (5 ASA), corticosteroids, and immunomodulatory medications are presently available and used. Meanwhile, diarrhea and abdominal pain are reported as common 5 ASA side effects. Likewise, long-term corticosteroid use frequently results in serious side effects such as bone loss, infection, diabetes, muscle wasting, and psychiatric disturbances.

Immunomodulatory drugs suppress the immune system, which regulates inflammatory bowel disease (IBD) symptoms. However, the resulting immunocompromised state makes the patient vulnerable to a wide range of diseases.

Coloectomy-based colitis surgery lowers life expectancy and increases the risk of complications.

In desert and semi-desert areas, camel milk accounts for 37% of the total milk source, 35% of sheep's milk, 23% of goat's milk and 10% of milk, making it one of the major sources of milk in these areas. Camel milk is rich in nutrients, having a content and structure better than other animal milk. The content of protein in camel milk is about 2-5.5%. The total protein content is similar to that of milk. The composition of casein is also similar to that of milk, but the proportion of each component is different from that of milk. The distribution range of most casein particles is obvious. It is wider than milk casein particles and has a larger diameter. The content of whey protein is higher, with no lactoglobulin or very low content. Accordingly, camel milk is more suitable for human metabolism.

In recent years, increasing attention has been paid to the health care function of fermented dairy products. The research on bioactive factors in fermented milk is more active.

Studies have shown that milk-derived hypotensive peptides can be extracted from milk protein and fermented milk products. At present, the antihypertensive effect of fermented dairy products has become a research hotspot. The hypotensive effect of lactic acid bacteria is that it can metabolize and produce protease to degrade proteins and produce active peptides. The inhibitory activity of the peptide is closely related to the type of lactic acid bacteria used. Different lactic acid bacteria fermentation processes have a variety of metabolites, protein hydrolysates are not the same, and fermented dairy product activity is also different. Therefore, the screening of probiotics with high production of desirable components is one of the directions for the development of lactic acid bacteria products and has broad prospects.

Camels have been an important means of transport for nomadic people in desert areas since ancient times, and camel milk is an important dairy source for dairy farmers living in desert and semi-desert areas. It is reported that camel milk is rich in protein, unsaturated fatty acids, vitamins and minerals essential to the human body. Its nutritional value is second only to human milk, higher than horse milk, goat milk, sheep milk and cow milk. People who consume camel milk, long term, have found some small impacts on tuberculosis, gastrointestinal diseases, kidney disease and cardiovascular disease as an adjuvant treatment.

In addition to containing milk and milk similar nutritional components, camel milk also contains a lot of antimicrobial agents such as lysozymes, lactoferrin and immunoglobulin factors in the body resistance mechanisms. Naturally fermented camel milk has a higher nutritional content and higher protein and fat content than fresh camel milk, making it a good food for the body to supplement protein and essential fatty acids. It not only retains all the nutrients of fresh milk, but also the fermentation of lactic acid to make the protein into a fine curd, thereby enhancing digestion and absorption rate. Further, some lactic acid bacteria fermentation processes can synthesize vitamin C, so naturally fermented camel milk vitamin C content is also higher than fresh camel milk.

The literature shows that the current research on the processing of camel milk is mainly directed to the nutritional value of camel milk, fermented camel milk processing, production of camel milk cream, and the production of water camellia cheese. However, research relating to the processing of camel milk for its treatment effects is rare.

Thus, an effective treatment for various bowel disorders with minimal side effects solving the aforementioned problems is desired.

### SUMMARY OF THE INVENTION

The present subject matter relates to food industry products that promote gut health and can be used to treat and/or prevent various diseases or disorders, such as inflammatory bowel disease, ulcerative colitis, Crohn's disease and bowel disorders (BD).

In one embodiment, the present subject matter relates to the use of fermented camel milk with a propolis-engineered probiotic for treating adverse BD. A propolis component, such as a honey propolis component, can be used as a synergistic supplement to engineer the probiotic bacterium, Enterococcus faecium (EF), at a defined bacterial load, propolis concentration and incubation period. The engineered EF can be added to fermented camel's milk as a promising treatment for BD.

In one embodiment, the present subject matter relates to a probiotic composition comprising: Enterococcus faecium at a concentration of about 1×10⁶ to about 1×10⁹ CFU/ml; about 0.5% to about 5% by weight of a honey propolis component; and about 5% to about 20% of camel milk. In a further embodiment in this regard, the honey propolis component can be pinocembrin.

Also contemplated herein is a method of treating or preventing a bowel disorder in a patient, comprising administering to a patient in need thereof a therapeutically effective amount of the herein described compositions. Further contemplated methods include a method of maintaining or promoting a healthy gut microflora, reducing digestive process toxic effects, stimulating a digestive system, or improving bowel control in a patient, comprising administering to a patient in need thereof a therapeutically effective amount of the herein described compositions.

In a further embodiment, the present subject matter relates to a method of producing a probiotic composition comprising: pasteurizing camel milk; adding a honey propolis component to the pasteurized camel milk to form a mixture; incubating the mixture with an Enterococcus faecium bacterium; fermenting the incubated mixture; and obtaining the probiotic composition.

These and other features of the present subject matter will become readily apparent upon further review of the following specification.

### BRIEF DESCRIPTION OF DRAWINGS

FIG. 1 is a chart showing the toxicity of propolis on probiotic survival capability.

FIG. 2 is a chart showing the effects of different concentrations of probiotics with different doses of honey propolis components on lipopolysaccharide (LPS) induced RAW 264.7 macrophage cell lines.

### DETAILED DESCRIPTION

The following definitions are provided for the purpose of understanding the present subject matter and for construing the appended patent claims.

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

“Subject” as used herein refers to any animal classified as a mammal, including humans, domestic and farm animals, and zoo, sports, and pet companion animals such as household pets and other domesticated animals such as, but not limited to, cattle, sheep, ferrets, swine, horses, poultry, rabbits, goats, dogs, cats and the like.

“Patient” as used herein refers to a subject in need of treatment of a condition, disorder, or disease, such as an acute or chronic airway disorder or disease.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

### Compositions

In one embodiment, the present subject matter relates to a probiotic composition comprising: Enterococcus faecium (EF) at a concentration of about 1×10⁶ to about 1×10⁹ CFU/ml; about 0.5% to about 5% by weight of a honey propolis component; and about 5% to about 20% of camel milk. In a further embodiment in this regard, the honey propolis component can be pinocembrin.

In an additional embodiment, the probiotic composition can comprise Enterococcus faecium at a concentration of about 1×10⁷ to about 1×10⁹ CFU/ml; about 1% to about 3% by weight of a honey propolis component; and about 5% to about 20% of camel milk.

In another embodiment, the probiotic composition can comprise Enterococcus faecium at a concentration of about 1×10⁸ to about 1×10⁹ CFU/ml; about 0.5% to about 2% by weight of a honey propolis component; and about 5% to about 20% of camel milk.

In still another embodiment, the probiotic composition can comprise Enterococcus faecium at a concentration of about 1×10⁸ to about 1×10⁹ CFU/ml; about 0.75% to about 1.5% by weight of a honey propolis component; and about 5% to about 20% of camel milk.

In yet other embodiments, the probiotic composition can comprise honey propolis as honey extract at a concentration of about 1 to 4%, about 1.5%, or about 4%, and/or an Enterococcus faecium concentration of 1×10⁶ concentration or 1×10⁷.

In certain embodiments, the probiotic composition can be a food, a food supplement, or any other suitable composition intended for oral administration. One specific embodiment of the present compositions is a food supplement. Similarly, the present compositions can be formulated as a solid, a semi-solid, a liquid, a solution, a suspension, or any other suitable composition for oral administration. In one specific embodiment, the present compositions can be formulated as a solid. Such solid compositions included herein can include tablets, capsules, sachets, powders, lyophilized powders, and the like. One specific embodiment of the present compositions is where the composition is formulated as a lyophilized powder.

In another embodiment, the present compositions can be provided as a sachet product. In this regard, the sachet can contain probiotic strains with honey propolis enriched with pinocembrin in fermented camel milk. This results in a prepared fermented product. The product can be activated by addition to water and incubating for about 3 to 8 hours.

In an embodiment, the present compositions can be prepared as foods for humans or other animals, including medical foods, functional foods, special nutrition foods and dietary supplements. A “medical food” is a product prescribed by a physician that is intended for the specific dietary management of a disorder or health condition for which distinctive nutritional requirements exist and may include formulations fed through a feeding tube (referred to as enteral administration or gavage administration). Yogurt is one specific example of a food that can be used according to the present compositions.

A “dietary supplement” shall mean a product that is intended to supplement the human diet and may be provided in the form of a pill, capsule, tablet, sachet, powder, or like formulation. By way of non-limiting example, a dietary supplement may include one or more of the following dietary ingredients: vitamins, minerals, herbs, botanicals, amino acids, and dietary substances intended to supplement the diet by increasing total dietary intake, or a concentrate, metabolite, constituent, extract, or combinations of these ingredients, not intended as a conventional food or as the sole item of a meal or diet. Dietary supplements may also be incorporated into foodstuffs, such as functional foods designed to promote control of glucose levels. A “functional food” is an ordinary food that has one or more components or ingredients incorporated into it to give a specific medical or physiological benefit, other than a purely nutritional effect. “Special nutrition food” means ingredients designed for a particular diet related to conditions or to support treatment of nutritional deficiencies.

Further, Enterococcus faecium fermented camel milk may be enriched with nutraceuticals and further potential probiotics according to the present subject matter.

Other components well known to be used in probiotic compositions, such as yogurt or other fermented dairy compositions, can optionally be further included in the present compositions. Certain non-limiting examples of such other components include sweet whey, calcium caseinate, purified cellulose, aspartame, sodium aluminum silicate, and the like.

Further suitable compositions may be formed by the incorporation of any normally employed excipients, such as, for example, pharmaceutical grades of mannitol, lactose, starch, magnesium stearate, talcum, cellulose derivatives, sodium crosscarmellose, glucose, sucrose, magnesium carbonate, sodium saccharin, talcum and the like.

### Methods of Use

In another embodiment, the present subject matter relates to a formulation of fermented camel's milk with a propolis-engineered probiotic for treating and/or preventing adverse bowel disorders (BD) in a patient. In one embodiment in this regard, the present subject matter relates to a method of treating or preventing a bowel disorder in a patient, comprising administering to a patient in need thereof a therapeutically effective amount of the herein described compositions. Further contemplated methods include a method of maintaining or promoting a healthy gut microflora, reducing digestive process toxic effects, stimulating a digestive system, or improving bowel control in a patient, comprising administering to a patient in need thereof a therapeutically effective amount of the herein described compositions.

In certain embodiments, the bowel disorder treatable herein can be selected from the group consisting of functional constipation, functional diarrhea, irritable bowel syndrome (IBS), ulcerative colitis, Crohn's Disease, inflammatory bowel disease, and combinations thereof. In further embodiments, the irritable bowel syndrome can be selected from the group consisting of constipation predominant IBS, alternating IBS, diarrhea predominant IBS, and combinations thereof.

In additional embodiments, administration of the herein described composition can reduce rectal bleeding and mucosal damage in the patient.

In this regard, present subject matter includes the use of fermented camel milk with propolis-engineered Enterococcus faecium at a desired microbial load. Camel milk fermented with Enterococcus faecium has not previously been used in large quantities to treat BD conditions. Further, the contemplated honey propolis component can have antibacterial, antiviral, antifungal, anti-inflammatory, antioxidant, immunostimulating, anti-tumor, and/or anti-Helicobacter pylori activity, yet has never previously been used to engineer Enterococcus faecium. Accordingly, the present subject matter introduces the synergistic complexion of a honey propolis component (at, for example, 0.5 to 5%) as a supportive growth medium for Enterococcus faecium that may manipulate cell membrane components.

The presently described methods can cause a substantial clinical recovery and reduction in severity of BD symptoms in a patient in a short period. By way of non-limiting example, after two weeks of treatment with the present compositions, a significant reduction in rectal bleeding and mucosal damage score may be observed. Furthermore, the levels of immune-modulatory markers such as, by way of non-limiting example, TNF α, VEGF-1, IL-1β, IFN-γ IFN T γ-6 and TGF-β, may also be achieved following administration of the present compositions to a patient, further supporting the positive recovery effects.

In another embodiment, the present subject matter includes the usage of supplemented camel milk with high bio absorption and accumulation of Enterococcus faecium in host gut systems for faster regeneration of bowel injury. This is the first finding about the usage of honey propolis to manipulate Enterococcus faecium to ferment camel's milk to be used as an effective and biocompatible formulation to treat BD.

### Methods of Production

In a further embodiment, the present subject matter relates to a probiotic composition comprising Enterococcus faecium incubated with a prebiotic component Propolis (Pinocembrin) which allows for a better survival of bacterial strains during storage. The compositions produced according to the present methods can achieve a positive probiotic effect, as well as enhanced probiotic survival in the gut, in use.

In this regard, in one embodiment, the present subject matter relates to a method of producing a probiotic composition comprising: pasteurizing camel milk; adding a honey propolis component to the pasteurized camel milk to form a mixture; incubating the mixture with an Enterococcus faecium bacterium; fermenting the incubated mixture; and obtaining the probiotic composition. In this regard, the honey propolis component can be enriched with pinocembrin.

When making the presently described Enterococcus faecium camel milk product, the content of Enterococcus faecium subspecies can result in a beneficial flora inoculum concentration of 0.1 DCU/1 kg.

### EXAMPLES

The following examples relate to certain compositions as described herein, and methods for making the same.

### Example 1—Procedure for Making the Present Compositions

1 cubic liter of fresh camel milk was pasteurized and heated to 75° C. for 10 min. The pasteurized milk was supplemented with honey propolis enriched with pinocembrin and stirred for 1 minute for uniform preparation. Fresh probiotic inoculum 0.1 DCU was added to the 1 liter of camel milk and stirred for 1 minute. The milk was left alone for 8 hours at 30° C., i.e., overnight, and the resultant product was collected as a probiotic supplemented camel milk product.

### Example 2—Specific Composition

An exemplary composition was prepared having the following formulation:

| Ingredient | % by Weight |
|---|---|
| Nonfat dried camel milk yogurt | 35.88 |
| Sweet Whey | 18.42 |
| Enterococcus faecium | 0.0016 |
| Calcium caseinate | 13.95 |
| Purified cellulose | 3.99 |
| Honey propolis (dry) | 5.01 |
| Aspartame (1 to 10 mixture with whey powder) | 5.58 |
| Sodium aluminum silicate | 0.33 |

This composition was prepared according to the general method described in Example 1, above. In this regard, the following steps were taken, in the following order: pasteurization, inoculum preparation, and addition of enriched supplement. To make a Honey enriched probiotic supplemented yogurt drink, the above ingredients were blended at room temperature.

### Example 3—Sachet Product

A package was provided having 1 sachet comprising 2 g freeze dried Enterococcus faecium (1×10¹⁰ cfu) and 2-3% honey propolis in 12-15% pasteurized camel milk. The sachets are dissolved in approximately 200 ml of drinking water just before consumption to provide 1 serving per day.

The following examples relate to methods of using the compositions as described herein.

### Example 4—Toxicity of Propolis on Probiotic Survival

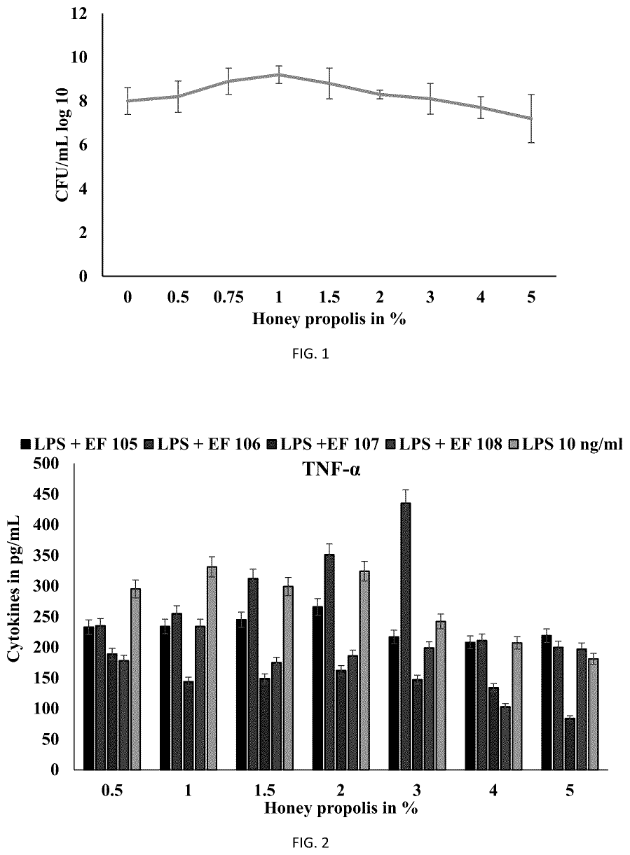

FIG. 1 describes the toxicity of propolis on probiotic survival capability. The initial concentration of the probiotic is 10⁸ and CFU was calculated after 6-hours incubation with different concentrations of honey propolis (0.5 to 5%). The concertation of honey propolis at 0.75 to 1.5% significantly improved the survival of probiotic Enterococcus faecium and cell viability. The gradual increasing concentration inhibited the bacterial growth with 5% inhibiting 11% of cell viability.

### Example 5—Effect on Macrophage Cell Lines

FIG. 2 describes how different concentrations of the probiotic with different doses of honey propolis on lipopolysaccharide (LPS) induced RAW 264.7 macrophages cell lines were evaluated. The highest inhibition percentages of TNF-alpha production in RAW264.7 cells treated with honey propolis after LPS stimulation were 80% (from 224 pg/mL to 150-120 pg/mL of TNF-α) as honey extract from 1 to 4% concentration. Enterococcus faecium 10⁶ concentration was significantly sensitive and Enterococcus faecium 10⁷ concentration was sensitive at 1.5% and 4% honey propolis supplemented products.

It is to be understood that the fermented camel milk with propolis-engineered probiotic is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A probiotic composition consisting of:
Enterococcus faecium at a concentration of about 1×10⁶ to about 1×10⁹ CFU/ml;
about 0.5% to about 5% by weight of a honey propolis component;
about 5% to about 20% of camel milk;
sweet whey;
calcium caseinate;
purified cellulose;
aspartame; and
sodium aluminum silicate;
wherein the probiotic composition is formulated as a yogurt.

2. The probiotic composition of claim 1, wherein the honey propolis component is pinocembrin.

3. The probiotic composition of claim 1, having:
the Enterococcus faecium at a concentration of about 1×10⁷ to about 1×10⁹ CFU/ml;
about 1% to about 3% by weight of the honey propolis component; and
about 5% to about 20% of the camel milk.

4. The probiotic composition of claim 1, having:
the Enterococcus faecium at a concentration of about 1×10⁸ to about 1×10⁹ CFU/ml;
about 0.5% to about 2% by weight of honey propolis component; and
about 5% to about 20% of the camel milk.

5. The probiotic composition of claim 1, having:
the Enterococcus faecium at a concentration of about 1×10⁸ to about 1×10⁹ CFU/ml;
about 0.75% to about 1.5% by weight of the honey propolis component; and
about 5% to about 20% of the camel milk.

6.-15. (canceled)
